(19) United States
(12) Patent Application Publication
(10) Pub. No.: US 2018/0315489 A1
(43) Pub. Date: Nov. 1, 2018

(54) GRANULAR DICHOTOMOUS SCORING METHOD FOR MACHINE LEARNING IN HEALTHCARE

(71) Applicants: Mark Ellis Jaruzel, II, Farmington Hills, MI (US); Ryan Gordon Beale, Huntington Woods, MI (US)

(72) Inventors: Mark Ellis Jaruzel, II, Farmington Hills, MI (US); Ryan Gordon Beale, Huntington Woods, MI (US)

(73) Assignee: The Live Network, Inc., Farmington, MI (US)

(21) Appl. No.: 15/964,019

(22) Filed: Apr. 26, 2018

Related U.S. Application Data
(60) Provisional application No. 62/490,115, filed on Apr. 26, 2017.

(51) Int. Cl. G16H 10/20; G16H 50/30; G06F 3/0484; G06N 3/08; G16H 10/60

(52) U.S. Cl. CPC G16H 10/20; G16H 50/30; G16H 10/60; G06N 3/08; G06F 3/04847

(57) ABSTRACT

The invention pertains to the fields of healthcare services and machine learning. More particularly, the invention pertains to a computer-implemented system for and method of gathering patient attitudes, values, opinions, traits, indicators of health, and symptoms of distress via a user interface that includes a moveable element with high granularity that presents a series of dichotomous choices allowing a patient or other person to move the element across the range of available values. The disclosure also describes a method of transforming legacy psychometric items into a form that modifies the kind of data produced through their usage. This continuous, high-granularity, data generated by the patient interaction can be used to generate training and test sets in machine learning and to facilitate the AI-optimized assessment, diagnosis, and treatment of mental and emotional health and distress at a distance. The present invention is unlimited with regard to the type of patient entity or healthcare professional entity.

### REFERENCE TO RELATED APPLICATIONS

This application claims an invention which was disclosed in Provisional Application No. 62/490,115, filed Apr. 26, 2017, entitled “GRANULAR DICHOTOMOUS SCORING METHOD FOR HEALTHCARE”. The benefit under 35 USC § 119(e) of the United States provisional application is hereby claimed, and the aforementioned application is hereby incorporated herein by reference.

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM (EFS-WEB)

Not Applicable

### STATEMENT REGARDING PRIOR DISCLOSURES BY THE INVENTOR OR A JOINT INVENTOR

Not Applicable

### BACKGROUND OF THE INVENTION

### Field of the Invention

The invention pertains to the fields of healthcare services, assessment, and machine learning. More particularly, the invention pertains to a computer-implemented system for and method of gathering patient attitudes, values, opinions, traits, indicators of health, and symptoms of distress via a user interface that includes a moveable element with high granularity that presents a series of dichotomous choices allowing a patient or other person to move the element across the range of available values. The continuous, high-granularity, data generated by the patient interaction can be used to generate training and test sets in machine learning and to facilitate the AI-optimized assessment, diagnosis, and treatment of mental and emotional health and distress at a distance.

### Description of Related Art

A mental health disorder, also commonly referred to as a mental illness, is a pattern of mood, cognition, behavior, or personality that occurs in a person and is thought to cause distress or disability that is not a normal part of development or culture. Mental health disorders are quite common. In the United States, the American Psychiatric Association estimates that over 68 million Americans will meet diagnostic criteria for a psychiatric or substance use disorder in a given year. Studies in several English-speaking countries have suggested that over the course of the lifespan it is more common to meet the criteria for a mental health disorder than to not meet criteria for a mental health disorder. The costs associated with treated, undertreated, and untreated mental illnesses are extremely high with The World Economic Forum estimating that worldwide costs were $2.5 trillion for the year 2010.

Access to adequate assessment and care for mental health disorders is lacking in many parts of the United States. The Substance Abuse and Mental Health Services Administration (SAMHSA) estimates that fewer than 50% of adults meeting diagnostic criteria for a mental health disorder receive any treatment for that disorder. The combination of stigma, low provider density areas, and inadequate treatment resources presently complicates the practice of mental healthcare. One component that will contribute to more adequately addressing mental health concerns will be increased identification of existing concerns through quick and accurate diagnostic tools.

One commonly employed method of psychodiagnostics is to ask patients questions utilizing multiple choice questions. These questions can be simple, forced-choice, dichotomous items (i.e. True or False questions) or they can be several point items often called Likert-style items (i.e. Strongly Disagree, Disagree, Neutral, Agree, Strongly Agree). These kinds of 2 to 7 point items initially emerged prior to the advent of modern computers and were a part of early psychodiagnostic instruments that were typically hand scored such as the initial Minnesota Multiphasic Personality Inventory (MMPI).

The move from forced choice items where the respondent was only offered two choices to items where the respondent was offered 5 or 7 items offered a range of advantages in the assessment of opinions, behavior, traits, and distress. It allowed researchers and clinicians to mitigate the effects of social desirability bias because respondents might be more willing to endorse a response such as “Somewhat Agree” on a 5 or 7-point item than they might be to endorse an “Agree” response on a 2-point item. Likert-style items offered a wider range of possible scores in a comparable sized scale than would be possible with a 2-item scale (i.e. a scale comprised of 4 2-point items might have a total score range of 4, while a scale comprised of 4 5-point items might have a total score range of 16). Likert-style items also allowed greater power and flexibility in the statistical tests used to analyze the results offered.

While the move from 2-point scales to Likert-style scales offered several advantages, it was not without its drawbacks. One of the key drawbacks is that the quantitative interpretation of the results of scores from Likert-style items is problematic. Simple parametric tests are likely inadequate because it is not clear that there is an equal change in respondent-perceived value between each item on a multipoint scale (i.e. the move from “Neutral” to “Somewhat Agree” is not the same as the move from “Somewhat Agree” to “Strongly Agree”). Because the scales are not comprised of continuous data with a fixed interval, the statistical tests germane to working with interval and ratio data are not strictly speaking applicable. This has led to confusion in interpretation and the potential loss of descriptive power in statistical and clinical analyses.

Recent advances in artificial intelligence (AI) and machine learning (ML) have rapidly accelerated the pace at which computer systems can match or surpass basic human expertise in tasks as diverse as playing chess, recommending products, answering trivia questions, suggesting cancer treatments, and driving cars. Much of the gains in AI and ML have derived from the training of artificial neural networks (ANN) on large sets of labeled data and then using the trained ML system to make predictions from unlabeled data.

In one approach to ML in particular, ANNs trained on labeled data with weights updated via backwards propagation, the training times can be extremely long and/or costly. Methods to reduce the training times involved in training an ANN or to increase the accuracy of a trained ANN offer significant practical and economic advantages.

### SUMMARY OF THE INVENTION

The present invention provides systems and methods that allow for the generation of a plurality of dichotomous items with high granularity and continuous data. These items are then presented to patients so that they can communicate attitudes, values, opinions, traits, indicators of health, and symptoms of distress to the system and/or healthcare professional via a graphical user interface that includes a moveable element with high granularity. The continuous, high-granularity, data generated by the patient interaction is then used to generate training and test sets for an ANN that serves to facilitate the AI-optimized assessment, diagnosis, and treatment of mental and emotional health and distress at a distance.

The process begins with the identification of a plurality of dichotomous categories consisting of attitudes, values, opinions, traits, and symptoms of health and distress that are of relevance to clinical assessment, diagnosis, treatment, and prognosis. In some cases, these categories will be related to existing items on tests or screeners used in clinical or related practice. In some cases, these categories will be related to existing constructs in the clinical literature. In other cases, these categories will be novel categories suggested by additional research. These categories will serve as prototypes from which one or more dichotomous items are generated.

Once a sufficient list of categories has been identified and/or generated, they will be transformed into items suitable for implementation in a slider-based interface with high granularity and continuous movement. These items will consist of a question followed by a contrasting dichotomous pair. The anchor points will, by design, represent extremes. Some representative examples of the anchor points for the dichotomous pairs might include: “Always”/“Never” or “Completely Outgoing”/“Completely Reserved.”

These items will be rendered into a computer program with a graphical user interface such that the question will feature a line underneath it with the anchor points of the dichotomous pair at opposing ends. In the middle will be a movable graphical user interface element that a person can leave in the middle or slide to any point on the line via means of touch, gesture, computer mouse dragging, or similar interactions with the interface.

These items will be presented to relevant groups of interest (patients with particular diagnoses, persons with particular personality traits, etc.) and a plurality of scores for various items and collections of items will be identified that serve to predict phenomena that are of relevance to clinical assessment, diagnosis, treatment, and/or prognosis.

The continuous, high-granularity, data generated by the patient interaction will be used to generate training and test sets for an ANN that serves to facilitate the AI-optimized assessment, diagnosis, and treatment of mental and emotional health and distress at a distance. Given that the data is highly granular and continuous, it is very likely that the time required to train such an ANN and the precision, accuracy, and recall of the ANN so trained will differ from that of an ANN that has been trained on analogous items that are of a Likert-style (i.e. 5-point or 7-point).

Once an ANN has been trained, tested, and found to perform adequately given the required parameters, it will be utilized by professionals to assist them in their clinical practice. This will be done through the presentation of the scores and/or predictions to a patient and/or professional or other entity.

The present invention is not intended to be limiting in the nature of the entity that is the patient. It is expected that the present invention will be used by a diverse range of healthcare professionals and patients.

### BRIEF DESCRIPTION OF THE DRAWINGS

Further advantageous features of the present invention will become more apparent when the following detailed description is taken along with reference to the accompanying drawings in which:

FIG. 1 shows a representative example of granular dichotomous items presented via a graphical user interface according to one embodiment of the present invention.

FIG. 2 shows further details of a representative example of granular dichotomous items presented via a graphical user interface according to one embodiment of the present invention.

FIG. 3 shows an example of a classical forced-choice item of the kind often used in psychometric tests and other screening instruments.

FIG. 4 shows an example of how the classical forced-choice item depicted in FIG. 3 has been transformed to render it into a dichotomous item with high granularity.

FIG. 5 shows an example of a classical Likert-style item of the kind often used in psychometric tests and other screening instruments.

FIG. 6 shows an example of how the classical Likert-style item depicted in FIG. 5 has been transformed to render it into a dichotomous item with high granularity.

### DETAILED DESCRIPTION OF THE INVENTION

As discussed above, the present invention provides systems and methods that allow for the generation of a plurality of dichotomous items with high granularity and continuous data. These items are then presented to patients so that they can communicate attitudes, values, opinions, traits, indicators of health, and symptoms of distress to the system and/or healthcare professional via a graphical user interface that includes a moveable element with high granularity. The continuous, high-granularity, data generated by the patient interaction is then used to generate training and test sets for an ANN that serves to facilitate the AI-optimized assessment, diagnosis, and treatment of mental and emotional health and distress at a distance.

Embodiments of the present invention will sometimes involve the transformation of legacy forced-choice dichotomous items and Likert-style items into the more granular and continuous items described here. Exemplars of this process are given below. Embodiments of the present invention will sometimes involve the generation of novel items that are directly implemented into a granular and continuous item. Exemplars of how these might look in some embodiments of the present invention are also given below.

In any case, it is important to note that the functional nature of the generation of continuous, high-granularity, data is of paramount importance. Even seemingly identical items with regard to question content, will likely produce notably different results with regard to ANN training time, ANN precision, ANN accuracy, and ANN recall when implemented as a highly granular, continuous, item as compared with a comparable item implemented as a Likert-style item. Those sufficiently skilled in the arts involved will also readily recognize that the design elements in various embodiments of the present invention could be highly varied and yet still achieve functional equivalency.

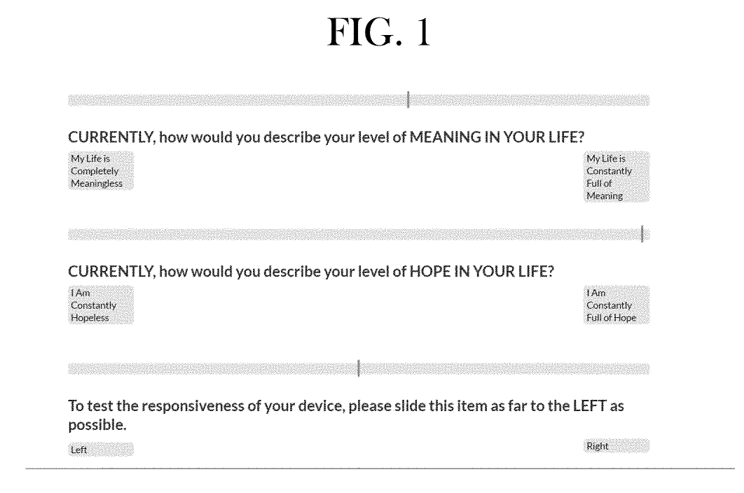

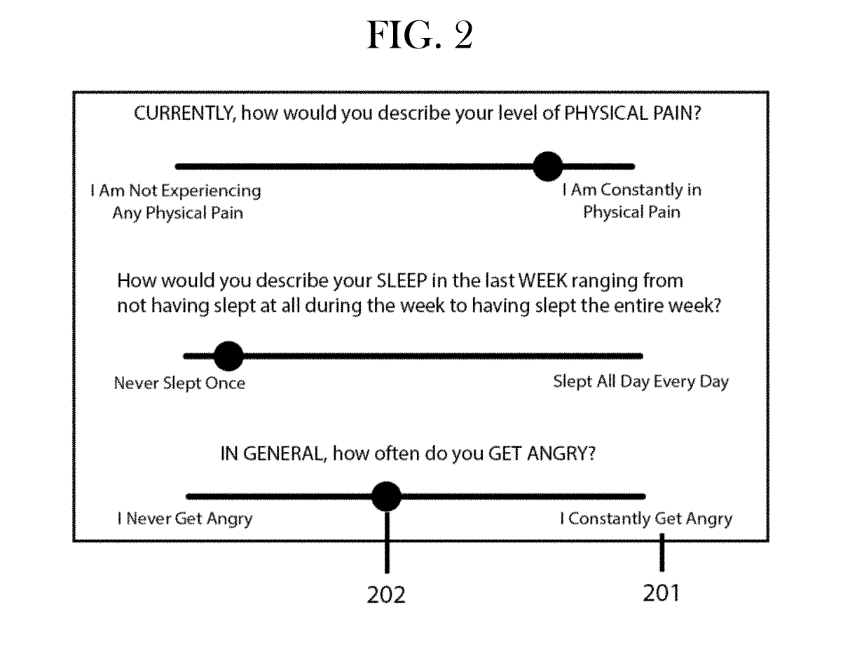

FIG. 1 illustrates several representative examples of granular dichotomous items presented via a graphical user interface according to one embodiment of the present invention. The specific features of these items and their functional implementation are elaborated upon in FIG. 2. The example graphical user interface could be comprised of hardware, software, or a combinational instance thereof that may be implemented in one or more computer systems or processing systems, whether local or cloud based, to carry out the functionality of the system as a whole. The interface could also be implemented as a physical device where a physical object is moved in instances where it may be advantageous (i.e. so that persons with difficulty seeing could provide granular answers).

FIG. 2 illustrates further details of a representative example of granular dichotomous items presented via a graphical user interface according to one embodiment of the present invention. Here dichotomous anchor points 201 are placed at either end of line or similar continuum with a terminus at or near the placement of the dichotomous anchor. A slider or moveable element 202 is presented starting midway between the dichotomous anchor points 201 and can be moved to any position within the range of available values. The final resting place of the moveable element 202 is recorded as a number and then stored in a computerized database along with other data from the same patient. In an ideal embodiment of the present invention, there are 500 points to each side of the starting place of the moveable element 202 and a single point that marks the initial starting place of this element for a total of 1,001 different positions on the line.

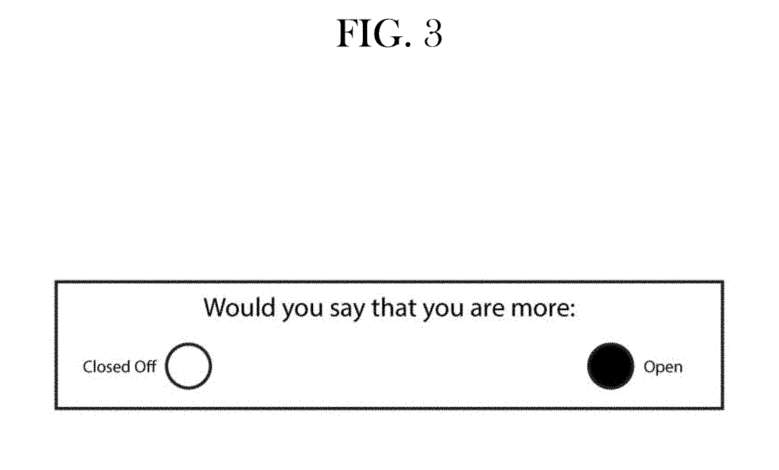

FIG. 3 illustrates an example of a classical forced-choice item of the kind often used in psychometric tests and other screening instruments. In this example, the person answering the question is forced to choose between only two options. Such an item can be problematic as the same scores would likely be obtained for individuals whom see themselves as extremely open, individuals whom seem themselves as slightly open, and individuals whom see themselves as equally open and closed off, but simply selected the open answer in order to move on to the next item. In ML programming, such an item might be coded as Closed=0 and Open=1. In such a case, all three respondents just discussed would be coded as “1” and they would have the same result stored in a database on their behalf. In ML, this question might disadvantageously lead to three different groups of respondents being clustered together as a single homogenous class with their actual heterogeneity being missed in the process.

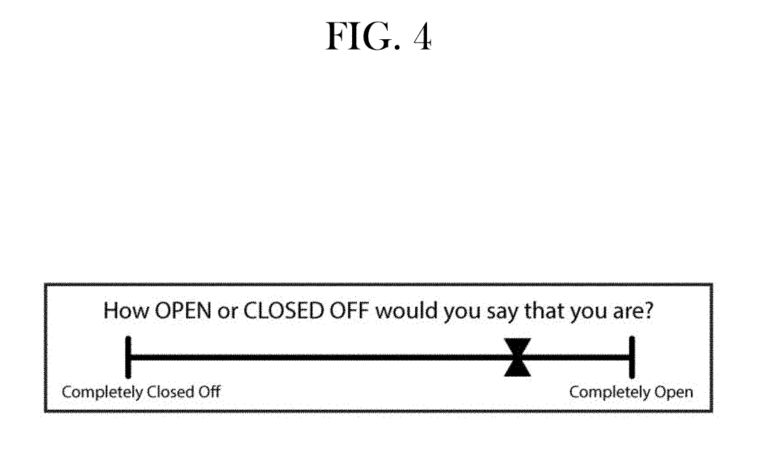

FIG. 4 illustrates an example of how the classical forced-choice item depicted in FIG. 3 has been transformed to render it into a dichotomous item with high granularity. In this example, the person answering the question is not forced to choose between only two options. Such an item could be less problematic than the item depicted in FIG. 3 as different scores would likely be obtained for individuals whom see themselves as extremely open, individuals whom seem themselves as slightly open, and individuals whom see themselves as equally open and closed off. In ML programming, such an item might be coded as Completely Closed Off=0 and Completely Open=1,000. In such a case, the three respondents just discussed might be coded as “1,000,” “675,” and “500” respectively and they would each have different results stored in a database on their behalf. In ML, this question might advantageously lead to three different groups of respondents being clustered separately with their actual heterogeneity being rendered obvious in the process.

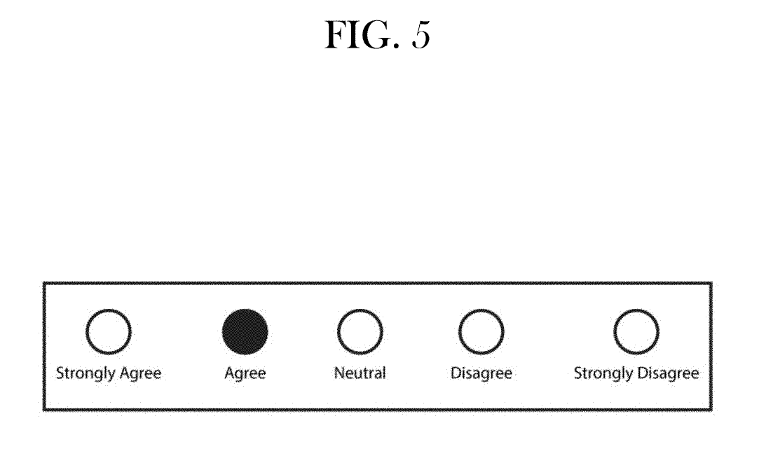

FIG. 5 illustrates an example of a classical Likert-style item of the kind often used in psychometric tests and other screening instruments. In this example, there is relatively more granularity than in the forced-choice item depicted in FIG. 3, but it is unclear if the intervals between each possible answer are the same. That is to say that the move from “Neutral” to “Agree” might not be the same as the move from “Agree” to “Strongly Agree.” This means that even if the item were coded “Strongly Agree”=5, “Agree”=4, “Neutral”=3, “Disagree”=2, and “Strongly Disagree”=1, certain descriptive statistics and parametric statistics would be questionable at best. In ML, an ANN could likely be trained on data comprised of such item assignments, but the ANN training time and ANN precision, ANN accuracy, and ANN recall when implemented in applications might be suboptimal.

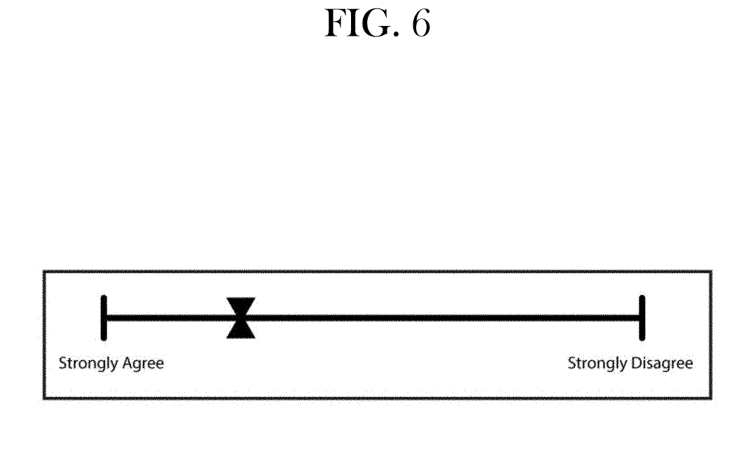

FIG. 6 illustrates an example of how the classical Likert-style item depicted in FIG. 5 has been transformed to render it into a dichotomous item with high granularity. Here, the new item is one that will yield mathematically more comprehensible data as the continuous nature of the data generated by the item means that the relationships between different points on the response line are clear as there is no ambiguity on the values of the intervals. In ML, an ANN trained on data comprised of items similar to FIG. 6 might result in a different ANN training time and different ANN precision, ANN accuracy, and ANN recall when implemented in applications as compared with an ANN trained on data comprised of items similar to FIG. 5.

Various user interfaces and embodiments were described above in some detail with reference to the drawings, wherein like reference numerals represented like parts and assemblies throughout the several views. Any of the preceding references to the various embodiments does not limit the scope of the claims attached hereto. Additionally, any examples set forth in this specification are not intended to be limiting and merely set forth some of the many possible embodiments for the appended claims. It is understood that various omissions and substitutions of equivalents are contemplated as circumstances may suggest or render expedient, but these are intended to cover applications or embodiments without departing from the spirit or scope of the claims attached hereto. Also, it is to be understood that any of the phraseology and terminology that were used herein were for the purpose of description and should not be regarded as limiting.

Any of the devices/servers/CPUs in the above-described systems may include a bus or other communication mechanism for communicating information, which interconnects subsystems and devices, such as processor, system memory (e.g., RAM), static storage device (e.g., ROM), disk drive (e.g., magnetic or optical), communication interface (e.g., modem or Ethernet card), display (e.g., CRT or LCD), input device (e.g., keyboard, touchscreen). The system component performs specific operations by the processor executing one or more sequences of one or more instructions contained in system memory. Such instructions may be read into system memory from another computer readable/usable medium, such as static storage device or disk drive. In alternative embodiments, hard-wired circuitry may be used in place of or in combination with software instructions to implement the invention. Thus, embodiments of the invention are not limited to any specific combination of hardware circuitry and/or software.

Any use of the word “screen,” “display,” or reference to a like concept above should be taken to mean a range of interfaces including but not limited to: a computer screen, a smartphone screen, a tablet screen, or an augmented reality screen or similar interface where a physical screen is lacking. Any references to a screen anywhere above are for the sake of brevity and should not be construed as a limitation on the types of devices or interfaces that can be utilized in various embodiments of this invention.

In an embodiment of the invention, execution of the sequences of instructions to practice the invention is performed by a single computing system. According to other embodiments of the invention, two or more computing systems coupled by a communication link (e.g., LAN, PTSN, or wireless network) may perform the sequence of instructions required to practice the invention in coordination with one another. The system component may transmit and receive messages, data, and instructions, including program, i.e., application code, through communication link and communication interface. Received program code may be executed by the processor as it is received, and/or stored in disk drive, or other non-volatile storage for later execution.

Various exemplary embodiments of the invention are described herein. Reference is made to these examples in a non-limiting sense. They are provided to illustrate more broadly applicable aspects of the invention. Various changes may be made to the invention described and equivalents may be substituted without departing from the true spirit and scope of the invention. In addition, many modifications may be made to adapt a particular situation, process, process act(s) or step(s) to the objective(s), spirit or scope of the present invention. Further, as will be appreciated by those with skill in the art that each of the individual variations described and illustrated herein has discrete components and features which may be readily separated from or combined with the features of any of the other several embodiments without departing from the scope or spirit of the present inventions. All such modifications are intended to be within the scope of claims associated with this disclosure.

Methods recited herein may be carried out in any order of the recited events which is logically possible, as well as in the recited order of events.

In addition, though the invention has been described in reference to several examples optionally incorporating various features, the invention is not to be limited to that which is described or indicated as contemplated with respect to each variation of the invention. Various changes may be made to the invention described and equivalents (whether recited herein or not included for the sake of some brevity) may be substituted without departing from the true spirit and scope of the invention. In addition, where a range of values is provided, it is understood that every intervening value, between the upper and lower limit of that range and any other stated or intervening value in that stated range, is encompassed within the invention.

Without the use of such exclusive terminology, the term “comprising” in claims associated with this disclosure shall allow for the inclusion of any additional element—irrespective of whether a given number of elements are enumerated in such claims, or the addition of a feature could be regarded as transforming the nature of an element set forth in such claims. Except as specifically defined herein, all technical and scientific terms used herein are to be given as broad a commonly understood meaning as possible while maintaining claim validity.

Accordingly, it is to be understood that the embodiments of the invention herein described are merely illustrative of the application of the principles of the invention. The breadth of the present invention is not to be limited to the examples provided, illustrated embodiments and/or the subject specification, but rather only by the scope of claim language associated with this disclosure.

### SEQUENCE LISTING

Not Applicable

What is claimed is:

1. An integrated computer-implemented system for allowing patients to communicate attitudes, values, opinions, traits, and symptoms of health and distress, facilitating patient assessment comprising:
A database configured to store application data (all internal and external programs required to run the system) and patient response data;
A plurality of dichotomous categories related to attitudes, values, opinions, traits, and symptoms of health and distress;
A display configured to receive patient input;
A display configured to display a graphical user interface that includes a moveable element with high granularity that presents dichotomous choices allowing a patient or other person to move the element across the available range of values via means of touch, gesture, computer mouse dragging, or similar interactions with the interface;
A computer-implemented processor configured to use artificial intelligence to analyze patterns, to transmit data to and receive data from patients and healthcare professionals across a range of devices and interfaces (including but not limited to: laptop computers, tablets, smart phones, mobile devices, augmented reality displays, wearables, and smart devices), and to transmit data to and receive data from a database.

2. The system of claim 1, wherein the patient is a person or other entity seeking professional consultation, education, assessment, diagnosis, intervention, or treatment.

3. The system of claim 1, wherein the database has been secured through encryption.

4. The system of claim 1, wherein the computer-implemented processor has been configured to use artificial intelligence (including but not limited to: deep learning, neural network modeling, parallel distributed processing, low-rank matrix factorization, regression analysis, vectorization, and skip thought vectors) to analyze patterns in patient data to initially suggest diagnosis and prognosis as well as advantageous and disadvantageous prescribed interventions for an individual patient.

5. The system of claim 1, wherein the patient interaction with the graphical user display elements can occur before, during, and/or after the rendering of professional services so serve such purposes as: initial assessment, cumulative assessment, summative assessment, diagnosis, feedback, prognosis, risk assessment, service or treatment matching, intervention matching, and provider matching.

6. A method, performed by a computer of the type having an image screen and a processor, for assessing attitudes, values, opinions, traits, and symptoms of health and distress in greater granularity, facilitating patient assessment with regard to initial assessment, cumulative assessment, summative assessment, diagnosis, feedback, prognosis, risk assessment, service or treatment matching, specific intervention matching, and optimal provider matching comprising the steps of:
Identifying a plurality of dichotomous categories consisting of attitudes, values, opinions, traits, and symptoms of health and distress that are of relevance to clinical assessment, diagnosis, treatment, and prognosis;
Identifying a plurality of scores for various items and collections of items that serve to predict clinically relevant phenomena that are of relevance to clinical assessment, diagnosis, treatment, and prognosis;
The presentation to the patient, via a screen or other graphical user interface, of a range of dichotomous choices allowing a patient or other person to move the element across the available range of values via means of touch, gesture, computer mouse dragging, or similar interactions with the interface;
The tabulation of scores generated via the range of values expressed by the patient or other person via the interface;
The use of a computer algorithm to make clinically relevant predictions based on the tabulated scores;
The presentation of the scores and/or predictions to a patient and/or professional or other entity.

7. A computer program product for use in conjunction with a computer device of the type having a processor and a screen, the computer program product comprising a computer readable, non-transitory, storage medium and instructions thereon (or a combinational equivalent of software and hardware whether embodied in a single device or a range of networked devices that is functionally equivalent) for enabling a professional to have improved information about patients and improved ability to tailor services and interventions to patients served where said program is comprised of the steps of:
Identifying a plurality of dichotomous categories consisting of attitudes, values, opinions, traits, and symptoms of health and distress that are of relevance to clinical assessment, diagnosis, treatment, and prognosis;
Identifying a plurality of scores for various items and collections of items that serve to predict clinically relevant phenomena that are of relevance to clinical assessment, diagnosis, treatment, and prognosis;
The presentation to the patient, via a screen or other graphical user interface, of a range of dichotomous choices allowing a patient or other person to move the element across the available range of values via means of touch, gesture, computer mouse dragging, or similar interactions with the interface;
The tabulation of scores generated via the range of values expressed by the patient or other person via the interface;
The use of a computer algorithm to make clinically relevant predictions based on the tabulated scores;
The presentation of the scores and/or predictions to a patient and/or professional or other entity.

8. A method, for transforming legacy forced-choice dichotomous items and Likert-style items into continuous, dichotomous, items with high granularity for the purposes of altering the training times, precision, accuracy, and recall of artificial neural networks (ANNs), comprising the steps of:
Identifying a plurality of legacy forced-choice dichotomous items and Likert-style items of relevance to clinical assessment, diagnosis, treatment, and prognosis;
Transforming them to render them into dichotomous items with two extreme anchor points;
The implementation of the new items in a computer program that presents them to a patient, via a screen or other graphical user interface, where the dichotomous anchor points are placed at either end of line or similar continuum with a terminus at or near the placement of the dichotomous anchor and a moveable element is presented starting midway between the dichotomous anchor points and can be moved to any position within the range of available values;
The final resting place of the moveable element is recorded as a number and then stored in a computerized database along with other data from the same patient.

9. The method of claim 8, wherein the range of available values is greater than 11.
